(19) United States
(12) Patent Application Publication
(10) Pub. No.: US 2025/0101061 A1
(43) Pub. Date: Mar. 27, 2025

(54) UPREGULATION OF FERRITIN HEAVY CHAIN 1 EXPRESSION

(71) Applicant: HBC Immunology Inc., Menlo Park, CA (US)

(72) Inventor: Bomi FRAMROZE, Portola Valley, CA (US)

(73) Assignee: HBC Immunology Inc., Menlo Park, CA (US)

(21) Appl. No.: 18/832,869

(22) PCT Filed: Feb. 3, 2023
(86) PCT No.: PCT/US2023/061975

§ 371 Date: Jul. 24, 2024

Related U.S. Application Data
(60) Provisional application No. 63/306,978, filed on Feb. 4, 2022.

(51) Int. Cl. C07K 7/06; A61K 38/00; A61P 7/06

(52) U.S. Cl. CPC C07K 7/06; A61P 7/06; A61K 38/00

(57) ABSTRACT

The present disclosure relates to isolated oligopeptides capable of increasing expression of ferritin heavy chain 1 (FTH1) by mammalian cells, as well as formulations thereof. The formulations are suitable for treating diseases or conditions associated with iron deficiency and/or anemia.

### CROSS-REFERENCE TO RELATED APPLICATIONS

This application claims priority to and the benefit of U.S. Provisional Application No. 63/306,978, filed Feb. 4, 2022, the content of which is incorporated herein by reference in its entirety.

### REFERENCE TO AN ELECTRONIC SEQUENCE LISTING

The content of the electronic sequence listing (197732000740SEQLIST.xml; Size: 18,684 bytes; and Date of Creation: Jan. 31, 2023) is herein incorporated by reference in its entirety.

### FIELD

The present disclosure relates to isolated oligopeptides capable of increasing expression of ferritin heavy chain 1 (FTH1) by epithelial cells, as well as formulations thereof. The formulations are suitable for treating diseases or conditions associated with iron deficiency and/or anemia.

### BACKGROUND

Protein powders mixed with milk, water or other beverages are popular dietary supplements are reported to help active adults improve physical performance by increasing muscle mass and strength (Pasiakos et al., Sports Med, 45:111-131, 2015). Also, protein supplements can be beneficial to athletes during endurance training and recovery from injury. Additionally, consumption of a salmon protein hydrolysate as a protein supplement is known to increase serum hemoglobin levels in patients suffering from iron-deficient anemia (Bomi et al., J Nutr Food Sci, 5:4, 2015).

Protein powders, however, are very complex compositions and supplements from some manufacturers may contain added sugars or toxic contaminants. Thus, what is needed in the art are isolated oligopeptides with desirable properties and formulations thereof. In particular, formulations enriched in bioactive oligopeptides are needed.

### BRIEF SUMMARY

The present disclosure relates to isolated oligopeptides capable of increasing expression of ferritin heavy chain 1 (FTH1) by epithelial cells, as well as formulations thereof. The formulations are suitable for treating diseases or conditions associated with iron deficiency and/or anemia.

### BRIEF DESCRIPTION OF THE DRAWINGS

FIG. 1 is an alignment of the amino acid sequence of the eight major oligopeptides from the three bioactive fractions FRP18, FRP20 and FRP30, along with a shared “EES” motif, a consensus sequence and sequence identifiers.

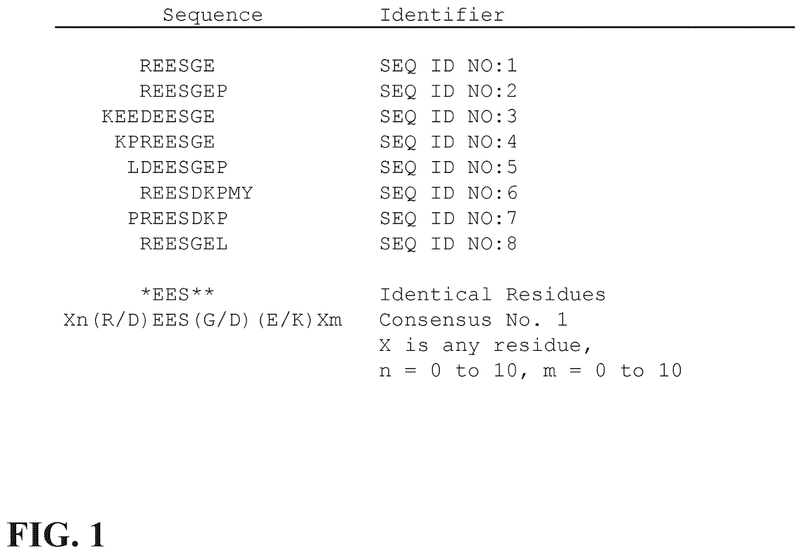

### DETAILED DESCRIPTION

The present disclosure relates to isolated oligopeptides capable of increasing expression of ferritin heavy chain 1 (FTH1) by epithelial cells, as well as formulations thereof. The formulations are suitable for treating diseases or conditions associated with iron deficiency and/or anemia.

### Definitions

As used herein and in the appended claims, the singular forms “a,” “or,” and “the” include plural references unless the context indicates otherwise. For example, “an excipient” includes one or more excipients.

It is understood that aspects and embodiments described herein as “comprising” include “consisting of” and/or “consisting essentially of” aspects and embodiments.

The term “about” as used herein in reference to a value describes from 90% to 110% of that value. For instance, about a 2-fold change in FTH1 mRNA expression includes a change of 1.8-fold to 2.2-fold, and includes 2.0-fold change in FTH1 mRNA.

As used herein, the terms “ferritin heavy chain 1” and “FTH1” refer to the nucleic acid sequence encoding the “ferritin heavy chain” protein, which is also known as the “ferritin H subunit.” The amino acid sequence of the human ferritin heavy chain is set forth as GenBank Accession No. NP_002023, and the mRNA sequence is set forth as GenBank Accession No. NM_002032, with the coding sequence extending from nucleotides 210-761.

The term “isolated” as used herein in reference to molecules (e.g., oligopeptides), refers to molecules that are removed or otherwise purified from their natural or synthetic environment. Substantially “isolated” molecules are at least 75% free, preferably at least 90% free, more preferably at least 95%, 96%, 97%, 98% or 99% free from other components. For instance, an “isolated oligopeptide consisting of the amino acid sequence of SEQ ID NO: 18” is at least at least 75% free of peptides and proteins that do not comprise the amino acid sequence of SEQ ID NO:18.

The term “increasing”, and grammatical equivalents as used herein in reference to expression or levels of FTH1 mRNA, refers to causing FTH1 mRNA to become greater in amount. Preferably, an increase in FTH1 mRNA encompasses a statistically significant increase, preferably an increase from about 2 to about 200 fold, from about 2 to 20 fold, from about 2 to 4 fold, preferably an increase of at least 2.0, 2.1, 2.2, 2.3, 2.4, 2.5, 3.0, 3.1, 3.2, 3.3, 3.4 or 3.5 fold.

As used herein, the terms “treating” and “treatment” refer to an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. As such, the terms “treating” and “treatment” as used herein, do not require complete alleviation of signs or symptoms, do not require a cure, and specifically include protocols that have a modest effect on the individual.

An “effective amount” of an agent disclosed herein (e.g., isolated oligopeptide or formulation thereof) is an amount sufficient to carry out a specifically stated purpose. An “effective amount” may be determined empirically in relation to the stated purpose. An “effective amount” or an “amount sufficient” of an agent is that amount adequate to affect a desired biological effect, such as a beneficial result, including a beneficial clinical result. The term “therapeutically effective amount” refers to an amount of an agent (e.g., isolated oligopeptide or formulation thereof) effective to “treat” a disease or disorder in a subject (e.g., a mammal such as a human). An “effective amount” or an “amount sufficient” of an agent may be administered in one or more doses.

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

### I. Isolated Oligopeptides

The isolated oligopeptides of the present disclosure consist of the amino acid sequence of Xm (R/D) EES (G/D) (E/K) Xn (Consensus No. 1), in which m and n are integers independently selected from the range of from 0-10, and each X, if present, is independently selected from any amino acid. Specifically, in some embodiments, the integers of m and n are each selected from 0, 1, 2, 3, 4, 5, 6, 7, 8, 9 or 10. Thus, the claimed oligopeptides are from 6 to 26 residues in length. In some embodiments, the oligopeptide is no less than 6, 7, 8, 9, 10, 11, 12, 13, 14, 15, 16, 17, 18, 19, 20, 21, 22, 23, 24 or 25 residues in length, and/or the oligopeptide is no more than 26, 25, 24, 23, 22, 21, 20, 19, 18, 17, 16, 15, 14, 13, 12, 11, 10, 9, 8, or 7 residues in length, in which the lower limit is less than the upper limit. In some embodiments, the isolated oligopeptide comprises the amino acid sequence of REESGE (SEQ ID NO:1). In some embodiments, the isolated oligopeptide comprises the amino acid sequence of REESGEP (SEQ ID NO: 2). In some embodiments, the isolated oligopeptide comprises the amino acid sequence of KEEDEESGE (SEQ ID NO:3). In some embodiments the isolated oligopeptide comprises the amino acid sequence of KPREESGE (SEQ ID NO:4). In some embodiments the isolated oligopeptide comprises the amino acid sequence of LDEESGEP (SEQ ID NO:5). In some embodiments, the isolated oligopeptide comprises the amino acid sequence of REESDKPMY (SEQ ID NO:6). In some embodiments, the isolated oligopeptide comprises the amino acid sequence of PREESDKP (SEQ ID NO:7). In some embodiments, the isolated oligopeptide comprises the amino acid sequence of REESGEL (SEQ ID NO:8). In some embodiments, the isolated oligopeptide comprises an amino acid sequence having at least 90% (e.g., at least 91%, 92%, 93%, 94%, 95%, 96%, 97%, 98%, 99%, or 100%) amino acid sequence identity to any one of SEQ ID NOS: 1-8. In some embodiments, the isolated oligopeptides of the present disclosure consist of the amino acid sequence of XjREESDKPXk (Consensus No. 2/SEQ ID NO:18), in which j is an integer selected from the range of from 0-10, k is an integer selected from the range of from 0-9, and each X, if present, is independently selected from any amino acid. Thus, in some embodiments, the claimed oligopeptides are from 7 to 26 residues in length. In some embodiments, the oligopeptide is no less than 7, 8, 9, 10, 11, 12, 13, 14, 15, 16, 17, 18, 19, 20, 21, 22, 23, 24 or 25 residues in length, and/or the oligopeptide is no more than 26, 25, 24, 23, 22, 21, 20, 19, 18, 17, 16, 15, 14, 13, 12, 11, 10, 9, or 8 residues in length, in which the lower limit is less than the upper limit. In some embodiments, the oligopeptides comprise the amino acid sequence of XgREESDKP (XhXi) (Consensus No. 3/SEQ ID NO:19), in which Xg is proline (P) or absent, XhXi is methionine and tyrosine (MY) or absent. In some embodiments, the isolated oligopeptide comprises the amino acid sequence of REESDKPMY (SEQ ID NO:6) or the amino acid sequence of PREESDKP (SEQ ID NO:7).

In preferred embodiments, the isolated oligopeptides are capable of increasing expression of ferritin heavy chain 1 (FTH1) mRNA by intestinal epithelial cells contacted with the oligopeptides. The increase in expression of FTH1 mRNA as a result of contact with the oligopeptide is relative to intestinal epithelial cells not contacted with the oligopeptide (e.g., baseline), relative to intestinal epithelial cells cultured under the same condition except for the absence of the oligopeptide, or except for the presence of a negative control oligopeptide (e.g., oligopeptide about the same length, but which does not comprise Consensus No. 1. In some embodiments, the intestinal epithelial cells are mammalian cells. In some preferred embodiments, the mammalian cells are human cells. In exemplary embodiments, the intestinal epithelial cells are HIEC-6 cells. In other embodiments, the intestinal epithelial cells are primary human intestinal epithelial cells.

In some preferred embodiments, the oligopeptide is produced synthetically. In exemplary embodiments, the oligopeptide is produced by solid phase synthesis and purified by high performance liquid chromatography as known in the art. Even so, the present disclosure also provides an isolated nucleic acid encoding the oligopeptide. The nucleic acid may be present in an expression cassette or vector in operable combination with a promoter. Also provided are host cells comprising the isolated nucleic acid, expression cassette or expression vector, for recombinant expression of the oligopeptide.

### II. Formulations

The present disclosure provides formulations comprising at least one isolated oligopeptide of the preceding section, and at least one pharmaceutically acceptable excipient and/or an oral delivery agent. In some embodiments, the formulations may further comprise an enteric coating, liposomes, microspheres, or micro-/nano-particles. For instance, in some embodiments, the isolated oligopeptide is encapsulated within an enteric coating, liposomes, microspheres, or micro-/nano-particles. However, as the oligopeptide of the formulation is isolated, the formulations of the present disclosure do not encompass fish protein hydrolysates, such as a salmon protein hydrolysate.

The amount of an oligopeptide of the disclosure, which will be effective in the treatment of a particular disorder or condition disclosed herein depends on the nature of the disorder or condition, and can be determined by standard clinical techniques. In addition, in vitro or in vivo assays may optionally be employed to help identify optimal dosage ranges. In some embodiments, the dose of the oligopeptide of the present disclosure is from about 0.1 mg/kg to about 1000 mg/kg, about 1.0 mg/kg to about 100 mg/kg, or about 10 mg/kg body weight of the subject to be treated. In some embodiments, the dose of oligopeptide is no less than 0.1, 0.5, 1.0, 5.0, 10, 20, 30, 40, 50, 60, 70, 80, 90, 100, or 500 mg/kg, and/or the dose of the oligopeptide is no more than 1000, 500, 100, 90, 80, 70, 60, 50, 40, 30, 20, 10, 5.0, 1.0, or 0.5 mg/kg, in which the lower limit is less than the upper limit.

### A. Excipients

Pharmaceutically acceptable excipients of the present disclosure include, for instance, solvents, bulking agents, buffering agents, tonicity adjusting agents, and preservatives (Pramanick et al., Pharma Times, 45:65-77, 2013). In some embodiments the formulations may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the formulations comprise an aqueous vehicle as a solvent. Suitable vehicles include for instance sterile water, saline solution, phosphate buffered saline, and Ringer's solution. In some embodiments, the formulation is isotonic.

The formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the formulation within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The formulations may comprise a tonicity adjusting agent. Suitable tonicity adjusting agents include for instance dextrose, glycerol, sodium chloride, glycerin and mannitol.

The formulations may comprise a bulking agent. Bulking agents are particularly useful when the pharmaceutical formulation is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The formulations may comprise a preservative. Suitable preservatives include for instance antioxidants and antimicrobial agents. However, in preferred embodiments, the formulation is prepared under sterile conditions and is in a single use container, and thus does not necessitate inclusion of a preservative.

### B. Oral Delivery Agents

Oral delivery agents of the present disclosure include, for instance, absorption enhancers, fatty acids, enzyme inhibitors, polyethylene glycol, mucoadhesive polymers, and cell penetrating peptides (Dan et al., Children, 7:307, 2020).

Commonly utilized routes of administration for therapeutic peptides and proteins include intravenous (IV), intraperitoneal (IP), and intramuscular (IM) injections. However, oral administration is preferred by patients and oral medications are typically less expensive to manufacture, distribute and administer. Unfortunately, development of orally available dosage forms of therapeutic peptides and proteins have been complicated for a variety of reasons, including but not limited to poor stability in physiological conditions, short biological half-life, and low permeability through the epithelial barrier in the small intestine. Thus, in some embodiments, the formulations of the present disclosure are designed to protect the isolated oligopeptide from the proteolytic enzymes and acidic environment found in the stomach, such that their bioactivity is retained as they are absorbed into the bloodstream (see, e.g., Dan et al., Children, 7:307, 2020).

### III. Methods of Use

The isolated oligopeptides and formulations of the present disclosure find use in methods and medicaments for increasing expression of FTH1 mRNA expression by mammalian cells. In some embodiments, the mammalian cells are epithelial cells, such as human epithelial cells. In some preferred embodiments, the isolated oligopeptides and formulations of the present disclosure find use in methods and medicaments for increasing serum ferritin concentration in a human subject in need thereof. In some preferred embodiments, the isolated oligopeptides and formulations of the present disclosure find use in methods and medicaments for treating or preventing a disease or condition in a human subject in need, wherein the disease or condition is associated with an inadequate serum ferritin concentration.

In some in vivo embodiments, the formulation is administered by mouth. For instance, the formulation may be administered enterically. In some embodiments, the formulation is administered by a buccal, a sublabial, or a sublingual route. In some embodiments, the mammalian subject (recipient) does not have cancer. In certain embodiments, the subject is a human subject that does not have prostate cancer. In some embodiments, the human subject is male. In other embodiments, the human subject is female.

Increasing FTH1 mRNA Expression.

In some aspects, the present disclosure provides methods and medicaments for increasing expression of ferritin heavy chain 1 (FTH1) mRNA in a mammalian subject in need thereof, comprising administering to the subject an effective amount of an oligopeptide or formulation of Section I or Section II. In some embodiments, the mammalian subject is a human subject.

Ferritin is a widely expressed and highly conserved protein and consists of 2 types of oligopeptide chains: ferritin heavy chain and ferritin light chain. Ferritin heavy chain catalyzes the Fe²⁺ oxidation reaction, whereas ferritin light chain plays an important role in the storage of Fe³⁺. Both chains are essential for maintaining iron homeostasis and preventing iron overload (Tian et al., Neurotherapeutics, 17:1796-1812, 2020). The FTH1 gene encodes the heavy subunit of ferritin. An increase in FTH1 expression, and thus ferritin, leads to an increase in iron absorption and bioavailability. This increase in iron absorption and bioavailability is beneficial to ameliorate diseases and disorders resulting from inadequate amounts of iron (e.g., iron deficient anemia).

In some embodiments, the methods for increasing expression of ferritin heavy chain 1 (FTH1) mRNA by mammalian cells, comprise contacting the mammalian cells with an effective amount of the formulation. In some embodiments, the method for increasing expression of FTH1 mRNA comprises contacting the mammalian cells with an effective amount of the formulation, wherein the increase is relative to baseline (e.g., pre-contact). In some embodiments, the method for increasing expression of FTH1 mRNA comprises contacting the mammalian cells with an effective amount of the formulation, wherein the increase is relative to mammalian cells not contacted with the formulation. In some embodiments, the method for increasing expression of FTH1 mRNA comprises contacting the mammalian cells with an effective amount of the formulation, wherein the increase is relative to mammalian cells contacted with a control formulation lacking the oligopeptide. In some embodiments, the method for increasing expression of FTH1 mRNA comprises contacting the mammalian cells with an effective amount of the formulation, wherein the increase is relative to mammalian cells containing a negative control oligopeptide.

In some embodiments, the contacting is in vivo. As such, in some embodiments, the methods and medicament for increasing ferritin heavy chain 1 (FTH1) expression in a mammalian subject in need thereof, comprise administering to the subject an effective amount of the formulation to increase FTH1 expression, wherein the increase is relative to baseline (e.g., pre-administration). In some embodiments, the methods and medicaments further comprise increasing serum ferritin concentration in a mammalian subject in need thereof. Thus, in some embodiments, the methods and medicaments for increasing serum ferritin concentration in a mammalian subject in need thereof, comprise administering to the subject an effective amount of the formulation to increase serum ferritin concentration, wherein the increase is relative to baseline (e.g., pre-administration).

### Treating or Preventing Iron Deficiency.

In some aspects, provided herein is a method of treating or preventing a disease or condition in a mammalian subject in need thereof, comprising administering to the subject an effective amount of the formulation to treat or prevent the condition. In some embodiments, the disease or condition is associated with iron deficiency.

Iron is essential to human and animal physiology. Insufficient iron in humans and animals can cause a number of diseases and disorders, including iron deficient anemia and stunted growth. Iron deficiency can be classified into two levels, iron deficiency without anemia and iron deficiency with anemia (IDNA and IDA, respectively), according to the hemoglobin measurement value. Iron deficiency without anemia (IDNA, also nonanemic iron deficiency) is usually insidious and a challenge for diagnosis and management (Zhu et al., Front Neur, 11:298, 2020). In some embodiments, the disease or condition to be treated or prevented is associated with iron deficiency without anemia. In other embodiments, the disease or condition to be treated or prevented is associated with iron deficiency with anemia.

### Treating or Preventing Anemia.

In some aspects, provided herein is a method of treating or preventing a disease or condition in a mammalian subject in need thereof, comprising administering to the subject an effective amount of the formulation to treat or prevent the disease or condition, wherein the disease or condition is associated with anemia.

In another embodiment, the disorder is iron deficient anemia. Anemia is a disorder characterized by a deficiency of red blood cells. Iron deficient anemia is anemia that is caused by an inadequate amount of iron. This iron deficiency may result from inadequate amounts of iron ingested, inadequate absorption of iron, blood loss, or a combination of these.

Anemia by iron deficiency can be distinguished from many other anemias. For example, iron deficient anemia is different from anemia resulting from chronic infectious, inflammatory, or malignant disorders, such as arthritis or cancer.

### Treating or Preventing Restless Leg Syndrome.

In some aspects, provided herein is a method of treating or preventing a disease or condition in a mammalian subject in need thereof, comprising administering to the subject an effective amount of the formulation to treat or prevent the disease or condition, wherein the disease or condition is restless leg syndrome (also referred to as restless legs syndrome).

Restless Legs Syndrome (RLS) is a common neurological disorder, and iron deficiency is thought to play a key role in its pathogenesis. Iron deficiency is common in RLS and iron deficiency anemia (IDA) is a well-known cause of secondary RLS. In addition, IDA is frequently observed in patients with RLS (Zhu et al., Front Neurol, 11:298, 2020). However, there is no known cure for RLS, and there is no one drug that works for every patient, although some individuals afflicted with RLS may find some relief with neuroactive drugs or iron supplementation (Mayo Clinic, 2020).

### Enumerated Embodiments

1. An isolated oligopeptide consisting of the amino acid sequence of Xm (R/D) EES (G/D) (E/K) Xn (Consensus No. 1), in which m and n are integers independently selected from the range of from 0-10, and each X, if present, is independently selected from any amino acid.
2. An isolated oligopeptide consisting of the amino acid sequence of XjREESDKPXk (SEQ ID NO:18), in which j is an integer selected from the range of from 0-10, k is an integer selected from the range of from 0-9, and each X, if present, is independently selected from any amino acid.
3. The isolated oligopeptide of embodiment 2, comprising the amino acid sequence of XgREESDKP (XhXi) (SEQ ID NO:19), in which Xg is proline or absent, XhXi is methionine and tyrosine or absent.
4. The isolated oligopeptide of embodiment 3, comprising the amino acid sequence of REESDKPMY (SEQ ID NO:6).
5 The isolated oligopeptide of embodiment 3, comprising the amino acid sequence of PREESDKP (SEQ ID NO:7).
6. The isolated oligopeptide of embodiment 1, comprising the amino acid sequence of REESGEL (SEQ ID NO:8).
7. The isolated oligopeptide of embodiment 1, comprising the amino acid sequence of REESGE (SEQ ID NO:1), REESGEP (SEQ ID NO:2), KEEDEESGE (SEQ ID NO:3), KPREESGE (SEQ ID NO:4), LDEESGEP (SEQ ID NO:5), REESDKPMY (SEQ ID NO:6), PREESDKP (SEQ ID NO:7), or REESGEL (SEQ ID NO:8).
8 The isolated oligopeptide of any one of embodiments 1-7, wherein the oligopeptide is capable of increasing expression of ferritin heavy chain 1 (FTH1) mRNA by mammalian cells contacted with the oligopeptide, optionally wherein the mammalian cells are human cells, and/or optionally wherein the mammalian cells are intestinal epithelial cells, skeletal muscle cells, astrocytes, or macrophages.
9. A formulation comprising the isolated oligopeptide of any one of embodiments 1-8, and at least one pharmaceutically acceptable excipient.
10. A formulation comprising the isolated oligopeptide of any one of embodiments 1-8, and an oral delivery agent.
11. The formulation of embodiment 10, wherein the oral delivery agent comprises an absorption enhancer, a fatty acid, an enzyme inhibitor, polyethylene glycol, a mucoadhesive polymer, a cell penetrating peptide, or a combination thereof.
12. The formulation of any one of embodiments 9-11, further comprising an enteric coating, liposomes, microspheres, and/or micro-/nano-particles.
13. An isolated nucleic acid encoding the oligopeptide of any one of embodiments 1-7.
14. An expression vector comprising the nucleic acid of embodiment 13 in operable combination with a promoter.
15. A host cell comprising the isolated nucleic acid of embodiment 13 or the expression vector of embodiment 14.
16. A medicament comprising the formulation of any one of embodiments 9-12.
17. A method for increasing expression of ferritin heavy chain 1 (FTH1) mRNA by mammalian cells, comprising contacting the mammalian cells with an effective amount of the formulation of any one of embodiments 9-12 to increase FTH1 expression.
18. The method of embodiment 17, wherein the mammalian cells are intestinal epithelial cells, skeletal muscle cells, astrocytes, or macrophages, and/or optionally wherein the mammalian cells are human cells, or optionally wherein the mammal cells are human intestinal epithelial cells.
19. The method of embodiment 17 or embodiment 18, wherein contacting is in vivo.
20. A method for increasing ferritin heavy chain 1 (FTH1) expression in a mammalian subject in need thereof, comprising administering to the subject an effective amount of the formulation of any one of embodiments 9-12 to increase FTH1 expression.
21. A method for increasing serum ferritin concentration in a mammalian subject in need thereof, comprising administering to the subject an effective amount of the formulation of any one of embodiments 9-12 to increase serum ferritin concentration.
22. A method for treating or preventing a disease or condition in a mammalian subject in need thereof, comprising administering to the subject an effective amount of the formulation of any one of embodiments 9-12 to treat or prevent the disease or condition.
23. The method of embodiment 22, wherein the disease or condition is associated with an iron deficiency.
24. The method of embodiment 22 or embodiment 23, wherein the disease or condition is associated with anemia.
25. The method of any one of embodiments 22-24, wherein the disease or condition is restless leg syndrome.
26. The method of any one of embodiments 20-25, wherein the formulation is administered by mouth.
27. The method of embodiment 26, wherein the formulation is administered enterically.
28. The method of embodiment 26, wherein the formulation is administered by a buccal, a sublabial, or a sublingual route.
29. The method of any one of embodiments 20-28, wherein the mammalian subject is a human subject.
30. The method of embodiment 29, wherein the human subject does not have cancer.

### EXAMPLES

Abbreviations: ACTB (beta-actin); FTH1 (ferritin heavy chain 1); H&E (hematoxylin and eosin); HSKMC (human skeletal muscle); LDH (lactate dehydrogenase); MBMM (mouse bone marrow-derived macrophage); RLS (restless leg syndrome); and SPH (salmon protein hydrolysate).

### Example 1

### Preparation of Biologically Active Salmon Protein Hydrolysate

Salmon Protein Hydrolysate (SPH) powder was produced by enzymatic hydrolysis of salmon (Salmo salar) head and backbone post filleting as described (US 2021/0252099). Briefly, 1000 grams of ground head and backbone was added to 1000 ml of water and the mixture was heated to 50° C. 10 g of an endopeptidase enzyme (pepsin) was added and the mixture was stirred for 30 minutes. Then 10 g of an exopeptidase enzyme (carboxypeptidase) was added and the mixture was stirred for 15 minutes. Next 5 grams of Flavourzyme® (a blend of endo- and exo-proteases derived from Aspergillus oryzae, marketed by Novozymes A/S, Bagsvaerd, Denmark) was added and the mixture was stirred for 10 minutes. The endopeptidase and exopeptidase treated salmon protein mixture was subsequently heated to 85° C. for 15 minutes to inactivate the proteases. After filtering, the hydrolysate fraction was concentrated to 30% dry matter in a conventional evaporator and spray-dried to yield salmon protein hydrolysate powder.

### Example 2

### Size Exclusion Fractionation of Salmon Protein Hydrolysate

A Dionex/Thermo UltiMate™ 3000 HPLC System (Thermo Fisher Scientific, Waltham, MA) equipped with a quaternary pump, an autosampler, an RS variate wavelength UV-Vis detector, and an automated fraction collector was used for preparative chromatography of the salmon protein hydrolysate (SPH). Separation was carried out at 25° C. on a BioSep™-SEC-s2000 size exclusion column (Phenomenex, Torrance, CA), 300×7.8 mm i.d., 145 Å pore size using TS software version 7.0.

The mobile phase consisted of 0.1 M phosphate buffer, pH 6.9. Isocratic elution was carried out using a flow rate of 5 ml/min for 48 min and monitored at 214 nm wavelength. An injection volume of 1 ml of aqueous solution of SPH (100 mg/ml) was used and twelve fractions (F1-F12) were collected from 4 to 48 minutes. Collected fractions were lyophilized and stored at −20° C.

### Example 3

### Regulation of Ferritin Heavy Chain 1 Expression by F1-F12 Fractions

An epithelial cell line derived from human small intestine was selected to assess the effect of SPH fractions on gene expression. This cell type was chosen since SPH has been studied as a gastrointestinal health modulator. HIEC-6 (CRL-3266™) cells obtained from ATCC (Manassas, VA) were propagated in OptiMEM™ 1 reduced serum medium supplemented with 20 mM HEPES, 10 mM GlutaMAX™, 10 ng/ml epidermal growth factor and fetal bovine serum to a final concentration of 4%, on 100 mm cell culture dishes. For this assay, the HIEC-6 cells were seeded in 24 well plates at a cell density of 1×10⁵ cells/cm² and maintained at 37° C. in a humidified 5% CO₂ atmosphere. About 24 hours later the cells were incubated with the SPH fractions F1-F12 each for 12 hours.

Total RNA was extracted using the UPzol reagent for the cell pellets, followed by a DNAse treatment (DNAse TURBO), using the manufacturer's protocols. Complementary DNA (cDNA) was synthesized with the High Capacity cDNA Reverse Transcription Kit (Applied Biosystems) using random hexamers. Gene expression levels were measured by qRT-PCR.

1 μl of cDNA corresponding to 50 ng of reverse transcribed RNA was amplified by Qper (QuantStudio™ 6 Flex Real-Time PCR System), using the TaqMan™ Universal PCR Master Mix (Catalog No. 4304437) and a TaqMan™ assay (Roche Molecular Systems, Inc., Pleasanton, CA).

FTH1 gene expression was estimated relative to expression of the housekeeping gene, beta-actin (ACTB), following the standard formula:
2-ΔCt. TaqMan™ probe IDs used were:
(i) FTH1, Hs01694011_s1; and (ii) ACTB, Hs01060665_g1. All experiments were run in triplicate.

As shown in Table 3-1, significant (>2 fold) upregulation of FTH1 gene expression was observed with F2 and F3 fractions. Non-significant (<2 fold) upregulation was observed with F7, F9 and F11 fractions.

TABLE 3-1
Effect of Treatment of HIEC-6 cells
with SPH fractions on FTH1 Expression

| Fraction | Gene | FTH1 Fold Change | | | Average | STD |
|---|---|---|---|---|---|---|
| F1 | FTH1 | 1.0 | 1.1 | 1.0 | 1.0 | 0.06 |
| F2 | FTH1 | 3.1 | 3.3 | 3.3 | 3.2 | 0.12 |
| F3 | FTH1 | 2.9 | 2.8 | 2.6 | 2.8 | 0.15 |
| F4 | FTH1 | 0.9 | 1.0 | 1.0 | 1.0 | 0.06 |
| F5 | FTH1 | 1.0 | 1.0 | 1.0 | 1.0 | 0.00 |
| F6 | FTH1 | 1.0 | 0.9 | 1.1 | 1.0 | 0.14 |
| F7 | FTH1 | 1.6 | 1.3 | 1.4 | 1.4 | 0.15 |
| F8 | FTH1 | 1.0 | 1.0 | 0.9 | 1.0 | 0.06 |
| F9 | FTH1 | 1.9 | 1.9 | 1.7 | 1.8 | 0.12 |
| F10 | FTH1 | 1.0 | 0.9 | 0.9 | 0.9 | 0.06 |
| F11 | FTH1 | 1.0 | 1.1 | 1.1 | 1.1 | 0.06 |
| F12 | FTH1 | 1.6 | 1.7 | 1.6 | 1.6 | 0.06 |

### Example 4

### Preparative Reversed-Phase Chromatography of F2 and F3 Fractions

Orthogonal chromatographic separation of combined F2 and F3 fractions was performed using the Dionex/Thermo UltiMate™ 3000 HPLC System (Thermo Fisher Scientific, Waltham, MA) described in Example 2. A 1 ml aliquot of an aqueous solution containing an equal weight of fractions F2 and F3 (100 mg/ml) were separated at 25° C. using a BetaSil™ C18 column (Thermo Fisher Scientific, Waltham, MA), 250×10 mm i.d., 10 micron particle size.

The mobile phase was water (solvent A) and acetonitrile (solvent B), both acidified with 0.05% of TFA at a flow rate of 4 ml/min. A gradient elution was carried out as follows: 0 min, 0% B; 10 min, 0% B; 45 min, 40% B; 50 min, 100% B; 60 min, 100% B.

The separation was monitored at 214 nm, and 12 fractions (FRP12 to FRP34) were collected from 12 to 36 minutes and evaluated for their FTH1 regulatory activity, as described in Example 3).

Only two early eluting fractions (at 18 and 20 minute retention times) and one late eluting fraction (at a 30 minute retention time) showed significant FTH1 gene upregulation as shown in Table 4-1. These three active fractions were labeled as FRP18, FRP20 and FRP30.

TABLE 4-1
Effect of Treatment of HIEC-6 cells with
SPH Sub-fractions on FTH1 Expression

| Retention | FTH1 |
|---|---|
| Time | fold |
| (min) | change |
| 12 | 1.1 |
| 14 | 1.0 |
| 16 | 0.9 |
| 18 | 3.1 |
| 20 | 3.3 |
| 22 | 1.6 |
| 24 | 1.1 |
| 26 | 1.4 |
| 28 | 1.5 |
| 30 | 3.4 |
| 32 | 1.1 |
| 34 | 1.2 |

### Example 5

### HPLC-HRMS Analysis of FRP18, FRP20 and FRP30 Fractions

Bioactive oligopeptides were identified using high performance liquid chromatography-high resolution mass spectrometry (HPLC-HRMS) analyses of freeze dried FRP18, FRP20 and FRP30 re-dissolved in 50% methanol. A reversed-phase Luna® Omega Polar C18 column (Phenomenex, Torrance, CA), 250×4.6 mm, 5 mm particles, 100 Å pore size with an injection volume of 10 μl on an Agilent 1290 chromatograph consisting of a G1340A degasser, a G1311A quaternary pump, a thermostatted column compartment, and a photodiode-array detector (Santa Clara, CA, USA) attached to a Bruker micrOTOF-Q II mass spectrometer equipped with an electrospray ionization interface.

The flow rate was maintained at 0.5 ml/min. The gradient elution profile of mobile phase A (water/acetonitrile 95:5 v/v) and mobile phase B (water/acetonitrile, 5:95 v/v), both acidified with 0.1% formic acid was: 0 min, 0% B; 5 min, 0% B; 25 min, 100% B; 35 min, 100% B; 37 min, 0% B.

Automated MS/MS spectra were acquired in positive ion mode, using a drying temperature of 200° C. and a drying gas flow of 8 l/minute. Structure identification was database-assisted using the MaxQuant software Version 2.0.3.0 and automated [M+H]n+ fragment matching analyses.

Eight major oligopeptides were identified from the three bioactive fractions FRP18, FRP20 and FRP30. Retention times, MS/MS results and oligopeptide sequences are shown in Table 5-1.

TABLE 5-1
Identification of Bioactive Oligopeptides

| | Retention | | | |
|---|---|---|---|---|
| | time | m/z, | | |
| Peak | (min) | [M + H]ⁿ⁺ | Sequence | Identifier |
| F18-1 | 7.6 | +1 803.8041 | REESGEP | SEQ ID NO: 2 |
| | | +2 402.4060 | | |
| | | +3 268.6066 | | |
| | | +4 201.7069 | | |
| | | +5 161.5671 | | |
| | | +6 134.8072 | | |
| F18-2 | 9.0 | +1 706.6874 | REESGE | SEQ ID NO: 1 |
| | | +2 353.8476 | | |
| | | +3 236.2344 | | |
| | | +4 177.4277 | | |
| | | +5 142.1438 | | |
| F20-1 | 9.9 | +1 931.9783 | KPREESGE | SEQ ID NO: 4 |
| | | +2 466.4931 | | |
| | | +3 311.3313 | | |
| | | +4 233.7505 | | |
| | | +5 187.2019 | | |
| | | +6 156.1696 | | |
| | | +7 134.0036 | | |
| F18-3 | 11.1 | +1 1155.2740 | REESDKPMY | SEQ ID NO: 6 |
| | | +2 578.1409 | | |
| | | +3 385.7632 | | |
| | | +4 289.5744 | | |
| | | +5 231.8611 | | |
| | | +6 193.3855 | | |
| | | +7 165.9030 | | |
| | | +8 145.2911 | | |
| F30-1 | 12.3 | +1 875.8646 | LDEESGEP | SEQ ID NO: 5 |
| | | +2 438.4362 | | |
| | | +3 292.6268 | | |
| | | +4 219.7220 | | |
| | | +5 175.9792 | | |
| | | +6 146.8173 | | |
| | | +7 125.9874 | | |
| F20-2 | 14.5 | +1 819.8469 | REESGEL | SEQ ID NO: 8 |
| | | +2 410.4274 | | |
| | | +3 273.9542 | | |
| | | +4 205.7176 | | |
| | | +5 164.7757 | | |
| | | +6 137.4810 | | |
| F20-3 | 15.2 | +1 958.0161 | PREESDKP | SEQ ID NO: 7 |
| | | +2 479.5120 | | |
| | | +3 320.0106 | | |
| | | +4 240.2599 | | |
| | | +5 192.4095 | | |
| | | +6 160.5092 | | |
| | | +7 137.7233 | | |
| F30-2 | 21.7 | +1 1051.9936 | KEEDEESGE | SEQ ID NO: 3 |
| | | +2 526.5007 | | |
| | | +3 351.3364 | | |
| | | +4 263.7543 | | |
| | | +5 211.2050 | | |
| | | +6 176.1721 | | |
| | | +7 151.1487 | | |
| | | +8 132.3811 | | |

### Example 6

### Effects of Oligopeptides on Human Skeletal Muscle Cells (HSKMC)

A cell line derived from human skeletal muscle was selected to assess the effect of SPH and oligopeptides on gene expression. HSkMC (PCS-950-010) obtained from ATCC (Manassas, VA) were propagated in Mesenchymal Stem Cell Basal Medium (ATCC) reduced serum medium supplemented with Primary Skeletal Cell Muscle Growth Kit (ATCC) that contains L-Glutamine (10 mM), Dexamethasone (10 M), rh epidermal growth factor (5 ng/ml), rh FGF-b (5 ng/ml), rh Insulin (25 μg/mL), and fetal bovine serum (4%), on 100 mm cell culture dishes. For this assay, the HSkMC cells were seeded in 24 well plates at a cell density of 1×10⁴ cells/cm² and maintained at 37° C. in a humidified 5% CO₂ atmosphere. About 24 hours later the cells were incubated with the SPH (100 μM) or a synthetic oligopeptide (10 μM) for about 12 hours.

Total RNA was extracted using the UPzol reagent for the cell pellets, followed by a DNAse treatment (DNAse TURBO), using the manufacturer's protocols. Complementary DNA (cDNA) was synthesized with the High Capacity cDNA Reverse Transcription Kit (Applied Biosystems) using random hexamers. Gene expression levels were measured by qRT-PCR.

1 μl of cDNA corresponding to 50 ng of reverse transcribed RNA was amplified by Qpcr (QuantStudio™ 6 Flex Real-Time PCR System), using the TaqMan™ Universal PCR Master Mix (Catalog No. 4304437) and a TaqMan™ assay (Roche Molecular Systems, Inc., Pleasanton, CA).

FTH1 gene expression was estimated relative to expression of the housekeeping gene, beta-actin (ACTB), following the standard formula:

2-ΔCt. TaqMan™ probe IDs used were:
(i) FTH1, Hs01694011_s1; and (ii) ACTB, Hs01060665_g1. All experiments were run in triplicate.

TABLE 6-1
Effect of Oligopeptides on FTH1
Expression in HSkMC

| Oligopeptide | | | Fold | | | |
|---|---|---|---|---|---|---|
| No./SEQ ID | Structure | Gene | Change | | | Average |
| SPH | mixed | FTH1 | 3.4 | 3.3 | 3.0 | 3.2 |
| | peptides | | | | | |
| FT-001 (NO: 1) | REESGE | FTH1 | 1.6 | 1.8 | 1.8 | 1.7 |
| FT-002 (NO: 2) | REESGEP | FTH1 | 2.1 | 2.0 | 2.5 | 2.2 |
| FT-008 (NO: 8) | REESGEL | FTH1 | 3.9 | 3.3 | 3.7 | 3.6 |
| FT-004 (NO: 4) | KPREESGE | FTH1 | 2.1 | 2.2 | 1.9 | 2.1 |
| FT-005 (NO: 5) | LDEESGEP | FTH1 | 1.7 | 1.8 | 1.8 | 1.8 |
| FT-003 (NO: 3) | KEEDEESGE | FTH1 | 1.1 | 1.0 | 1.3 | 1.1 |
| FT-006 (NO: 6) | REESDKPMY | FTH1 | 4.5 | 4.7 | 4.9 | 4.7 |
| FT-007 (NO: 7) | PREESDKP | FTH1 | 2.5 | 2.6 | 2.6 | 2.6 |

As shown in Table 6-1, appreciable (>2 fold) upregulation of FTH1 gene expression was observed when primary human muscle cells were incubated in the presence of FT-002, FT-004, FT-006, FT-007, and FT-008 peptides. Furthermore, dose-dependent effects on FTH1 gene expression were observed when HSKMC were treated with increasing oligopeptide concentrations, as shown in Table 6-2 below.

TABLE 6-2
Dose Response Effect of Oligopeptides
on FTH1 Expression in HSkMC

| | | Fold Change* | | | | |
|---|---|---|---|---|---|---|
| Oligopeptide | | 10 | 5 | 2.5 | 1.25 | 0.625 |
| No./SEQ ID | Structure | μM | μM | μM | μM | μM |
| SPH | mixed | 1.4 | 1.0 | 1.0 | 1.0 | 1.0 |
| | peptides | | | | | |
| FT-002 (NO: 2) | REESGEP | 2.2 | 1.4 | 1.1 | 1.0 | 1.0 |
| FT-008 (NO: 8) | REESGEL | 3.8 | 2.7 | 2.1 | 1.0 | 1.1 |
| FT-004 (NO: 4) | KPREESGE | 2.1 | 1.3 | 1.1 | 1.1 | 1.1 |
| FT-006 (NO: 6) | REESDKPMY | 4.9 | 4.2 | 3.5 | 2.2 | 1.3 |
| FT-007 (NO: 7) | PREESDKP | 2.8 | 1.9 | 1.1 | 1.0 | 1.1 |

*Average of triplicate experiments (SD ≤ 0.3)

### Example 7

### Effects of Oligopeptides on Human Astrocytes

This example describes assessment of protective effects of SPH and oligopeptides on hemin-induced iron-dependent oxidative injury of human astrocytes in vitro.

Immortalized human astrocytes (P10251-IM) were obtained from Innoprot, Spain and prepared under conditions recommended by the supplier. At 90% confluency, the P10251-IM cells were rinsed with 8 ml of DPBS and 2 ml of the T/E Solution (prepared as per Innoprot procedure), then added into an Erlenmeyer flask and gently rocked so that the P10251-IM cells would be completely covered. The flask was subsequently incubated at 37° C. for 2 minutes. Then 5 ml of fetal bovine serum (FBS) and the T/E solution from the flask were added into a 50 ml conical centrifuge tube. The flask was incubated for an additional 2 minutes, and the P10251-IM cells dislodged from the surface by gentle tapping. The P10251-IM cells were then transferred to the 50 ml conical centrifuge tube with 5 ml of TNS solution. The tube was centrifuged at 1000 rpm for 5 minutes. Then the P10251-IM cells were resuspended in the culture medium, counted, and plated in a poly-L-lysine-coated culture plate at 1×10⁴ cell density. Cells were tested after they were incubated for 21 days in vitro.

Confluent P10251-IM cell cultures were washed and then pre-treated with medium alone, or medium supplemented with SPH (160 μM) or an oligopeptide (10 μM) for 24 hr at 37° C. Commercial collagen peptide (Vital Protein) at 160 μM concentration and sterile water were used as negative controls. The P10251-IM cells were subsequently treated with 30 μM hemin in serum-free medium consisting of MEM with 10 mM glucose (MEM10) for 6 hrs.

P10251-IM astrocyte cultures that were pre-treated with 30 μM hemin sustained widespread cell injury. Cell viability was quantified using the lactate dehydrogenase (LDH) release assay as previously described (Chen and Regan, Curr Neurovasc Res, 2:189-196, 2005). Briefly, quantification of the protective effects of SPH and oligopeptides were assessed by determining the mean percentage LDH released, scaled to the complete lysis control culture which was treated with 0.1% Triton X-100 (considered to release 100% of culture LDH).

TABLE 7-1
Protection of Human Astrocytes From Hemin-Induced Injury

| Test Article | % LDH Release | | | Average | SD |
|---|---|---|---|---|---|
| 0.1% Triton X-100 | 100 | 99 | 100 | 100 | 0.6 |
| control | 48 | 49 | 46 | 48 | 1.5 |
| SPH | 45 | 43 | 43 | 44 | 1.2 |
| FT-006 | 40 | 39 | 42 | 40 | 1.5 |
| FT-008 | 29 | 27 | 31 | 29 | 2.0 |
| FT-007 | 35 | 38 | 34 | 36 | 2.1 |

L-Ferritin and H-Ferritin gene expression were then quantified. Briefly, after 6 hr incubation with hemin, the astrocytes were lysed, and RNA was extracted using the Qiagen lipid mini kit. The expression of both genes (L-ferritin and H-ferritin) involved in iron homeostasis was assessed by qRT-PCR. Fold changes were calculated using the ΔCt method, using GAPDH as the housekeeping gene. Fold changes were normalized to expression levels in control wells. Experiments were run in triplicate.

The primers used for amplification of cDNA were:

| | H-Ferritin Forward: |
|---|---|
| (SEQ ID NO: 10) | |
| | TAAAGAACTGGGTGACCACGTGAC; |
| | H-Ferritin Reverse: |
| (SEQ ID NO: 11) | |
| | AAGTCAGCTTAGCTCTCATCACCG; |
| | L-Ferritin Forward: |
| (SEQ ID NO: 12 | |
| | TGGCCATGGAGAAGAACCTGAATC; |
| | and |
| | L-Ferritin Reverse: |
| (SEQ ID NO: 13) | |
| | GCTTTCCAGGAAGTCACAGAGAT. |

TABLE 7-2
Effect of Oligopeptides on Ferritin Expression in Astrocytes

| Test Article | H-Ferritin Fold Change | | | Average | SD |
|---|---|---|---|---|---|
| Water | 1.0 | 1.0 | 1.0 | 1.0 | 0.00 |
| control | 1.0 | 1.0 | 1.1 | 1.0 | 0.06 |
| SPH | 2.4 | 2.3 | 2.4 | 2.4 | 0.06 |
| FT-006 | 2.9 | 2.9 | 3.1 | 3.0 | 0.12 |
| FT-008 | 4.1 | 4.4 | 4.1 | 4.2 | 0.17 |
| FT-007 | 3.5 | 3.2 | 3.3 | 3.3 | 0.15 |
| Test Article | L-Ferritin Fold Change | | | Average | SD |
| Water | 0.9 | 1.0 | 1.0 | 1.0 | 0.06 |
| control | 1.0 | 1.0 | 1.0 | 1.0 | 0.00 |
| SPH | 1.0 | 1.1 | 1.1 | 1.1 | 0.06 |
| FT-006 | 1.3 | 1.3 | 1.3 | 1.3 | 0.00 |
| FT-008 | 1.8 | 1.6 | 1.5 | 1.6 | 0.15 |
| FT-007 | 1.1 | 1.2 | 1.0 | 1.1 | 0.10 |

Ferritin over-expression and ferritin pretreatment has been reported to reduce hemin-induced oxidative toxicity in endothelial cells and astrocytes (Balla et al., J. Biol Chem, 267:18148-18153, 1992; and Regan et al., Neuroscience, 113:985-994, 2002). Now, SPH pre-treatment of astrocytes has been found to result in minor improvements in viability of hemin-injured astrocytes. Moreover, all three oligopeptides, FT-006, FT-007, and FT-008 (10 μM) have been found to provide substantial prophylactic protection of astrocytes from oxidative damage induced by hemin (i.e., 40%, 36%, and 29% mortality versus 48% mortality for the collagen control, respectively). Furthermore, all three oligopeptides showed a selective upregulation of the H-Ferritin gene, with little to no change in expression of the L-Ferritin gene. Specifically, oligopeptides FT-006, FT-007, and FT-008 resulted in increases in H-Ferritin gene expression of 3.0, 3.3, and 4.0 fold, respectively. These results indicate that protection of the astrocytes from hemin-induced iron-oxidative damage occurs at least in part via upregulation of expression of H-Ferritin gene.

### Example 8

### Effects of Oligopeptides on Murine Macrophages

This example describes the assessment of effects of SPH and oligopeptides on expression of H-Ferritin protein and mRNA expression in mouse bone marrow-derived macrophages (MBMMs) in normal, iron-enhanced, and iron-depleted cell culture conditions.

Cell Cultures. MBMMs (C57BL/6 genetic background) were obtained from CellBiologics.net and prepared under the conditions recommended by the supplier. Isolated MBMMs were seeded onto 10 cm diameter Petri dishes and grown in RPMI-GlutaMAX™ medium (Invitrogen) supplemented with 10% heat-inactivated FCS (Gibco), 10% L-cell-conditioned medium (source of colony-stimulating factor 1), 2 mM L-glutamine, 50 U/mL penicillin, and 50 mg/mL streptomycin. At 4 days after seeding, the adherent MBMMs were rinsed twice with Hanks balance salt solution (HBSS), and the medium was renewed for 7 days, at which point the MBMMs had completely differentiated into macrophages that were ready for downstream experimental applications.

Cell Treatments. SPH (average molecular weight of about 1100 Daltons) and oligopeptides were dissolved in sterile water to obtain 0.1 mM stock solutions. The SPH and oligopeptides were further diluted in cell culture media to reach final concentrations of 160 μM SPH and 10 μM oligopeptide per Petri dish.

Murine macrophages were incubated with SPH or an oligopeptide in the presence or absence of an iron-nitrilotriacetate solution (Fe-NTA; FeCl₃ (100 μM)-NTA (400 μM)) for 8 h at 37° C. to increase cellular iron concentration. Murine macrophages were also incubated with SPH or an oligopeptide in the presence or absence of desferrioxamine mesylate (DFO; 100 μM) for 16 h at 37° C. to decrease cellular iron concentration. Commercial collagen peptide (Vital Protein) at a 10 μM concentration and sterile water were used as negative controls.

Quantification of H-Ferritin. Macrophages were washed with cold saline and lysed with 400 μl of a solution containing 50 mM HEPES (Gibco), 1% IGEPAL C-630 (Sigma), and 1% proteases inhibitor cocktail P840 (Sigma). H-Ferritin concentrations in the lysates were determined by ELISA using polyclonal antibodies raised against mouse recombinant H-ferritin subunits and calibrated with the corresponding recombinant homopolymers. The specificity and the absence of cross-reactivity of the antibodies are documented. The results were expressed as (ng ferritin) per (mg total protein) in the cell lysate and shown as an average of triplicate readings in Table 8-1. Total protein content was measured using the BCA protein assay kit (Pierce).

Measurement of Gene Expression. Total RNA was extracted from macrophages using the Qiagen miRNeasy Mini Kit for purification of total RNA from animal and human cells and tissues according to the manufacturer's instructions. About 2 μg of total RNA was transcribed into cDNA, after RT2 RNA QC PCR Array was run to confirm the quality of the RNA samples followed by gene expression analysis using RT2 Profiler PCR array.

The primers used for amplification of cDNA were:

| | HPRT1 Forward: |
|---|---|
| (SEQ ID NO: 14) | |
| | 5′-GTAATGATCAGTCAACGGGGGAC-3′; |
| | HPRT1 Reverse: |
| (SEQ ID NO: 15) | |
| | 5′-CCAGCAAGCTTGCAACCTTAACCA-3′; |
| | FTH1 Forward: |
| (SEQ ID NO: 16) | |
| | 5′-GGAGTTGTATGCCTCCTAC-3′; |
| | and |
| | FTH1 Reverse: |
| (SEQ ID NO: 17) | |
| | 5′-GAGATATTCTGCCATGCC-3′. |

These primers do not co-amplify genomic DNA. All reactions were performed in a total reaction volume of 20 μl with iQ™ SYBR® Green Supermix (Bio-Rad) and carried out in the iQ™5 instrument (Bio-Rad). Baseline thresholds were calculated by Bio-Rad iQ5 program, and the threshold cycles (Ct) were used, wherein Ct values for the Fth1 gene were normalized to expression levels of Hprt1 (housekeeping) gene. Results are shown in Table 8-2 as n-fold difference relative to the control samples.

TABLE 8-1
Effect of Oligopeptides on H-Ferritin Protein
Levels\hat{ } in Murine Macrophages

| | | | Fe-NTA | | DFO | |
|---|---|---|---|---|---|---|
| Test Article | Fe-Normal | +/−SD | (+Fe) | +/−SD | (−Fe) | +/−SD |
| Water | 123 | 21 | 228 | 31 | 55 | 14 |
| SPH | 157 | 26 | 209 | 28 | 78 | 16 |
| FT-006 | 174 | 17 | 257 | 27 | 105 | 22 |
| FT-008 | 158 | 20 | 200 | 22 | 122 | 23 |
| FT-007 | 142 | 19 | 197 | 29 | 111 | 13 |

\hat{ }results shown as ng/mg total protein

TABLE 8-2
Effect of Oligopeptides on FTH1 Levels
in Murine Macrophages

| | | Fe-NTA | DFO |
|---|---|---|---|
| Test Article | Fe-Normal | (+Fe) | (−Fe) |
| Water | 1.0 | 2.9 | 0.4 |
| SPH | 2.1 | 3.1 | 1.8 |
| FT-006 | 2.7 | 2.8 | 3.3 |
| FT-008 | 2.5 | 2.7 | 3.4 |
| FT-007 | 1.9 | 2.9 | 3.1 |

The three most active oligopeptides were FT-008, FT-007, and FT-006. Treatment of murine macrophages with each of these three peptides resulted in substantial increases in FTH1 mRNA expression and in intracellular H-Ferritin protein levels in both normal and iron-depleted conditions. The H-Ferritin protein increase was most pronounced in the iron-depleted cellular environment, while little to no changes in FTH1 mRNA expression and H-Ferritin protein levels were observed in the iron-rich cellular environment.

### Example 9

### Analysis of Ferritin Concentration and Locomotion in Oligopeptide-Treated Mice

This example describes a murine model of restless leg syndrome (RLS) and its use for assessing the effect of oligopeptide treatment on concentrations of ferritin in serum and cerebral spinal fluid. This model is also suitable for assessing the effect of oligopeptide treatment on locomotion.

Animal model and treatment. C57/BL6 mice are fed with an iron deprivation ID diet (TD 80396, Harlan, WI, USA) containing only 3.5 mg iron/kg. One month after starting the ID diet, 6-hydroxydopamome (6-OHDA) lesions are made in the bilateral All nuclei. Briefly, the animals are anesthetized and then stereotaxically injected (medio-lateral 0.35 mm, dorsal/ventral 4.5 mm, antero-posterior-1.95 mm) with 1 μl of 0.2% 6-OHDA in 0.01% ascorbic acid saline into the bilateral All nuclei as previously described (Qu et al., J Neuropathol Exp Neurol, 66:383-388, 2007; and Luo et al., Sleep Medicine, 12:41-46, 2011).

Mice are then randomly assigned to various treatment groups (n=5-10 mice per group): (i) vehicle control, (2) SPH, (3) oligopeptide FT-006, (iv) oligopeptide FT-007, and (v) oligopeptide FT-008. Additional groups may be included for testing one or more of FT-002, FT-004, and other oligopeptides matching Consensus No 1. Oligopeptide doses for this study may be in the range of from about 1 mg/kg to about 1000 mg/kg, or from about 10 mg/kg to about 100 mg/kg body weight of the mice. After an initial treatment by intraperitoneal injection on Day 1, subsequent treatments are administered orally in mouse chow. The study may continue for up to 4, 8 or 12 weeks.

Locomotor activity measurement. Locomotor activity measurements are carried out using a Accu-Scan Digiscan system (Acuscan Instruments, Columbus, OH, USA). Data collected by computer includes Total Distance traveled (cm/60 min) and Moving Time (s/60 min). The measurements are carried out from 9 AM to 11 AM in a dark room. Each mouse is placed in the testing chamber for 30 min for adaptation, followed by a 60 min recording using the computer-generated automatic analysis system. The locomotor activities are measured in mice before treatment is initiated at Day 0 (baseline activity level) and then periodically through the course of the study.

Blood samples are collected (tail vein or cheek puncture) every two weeks for analysis of iron and biomarker levels. At the conclusion of the study, mice are humanely sacrificed. The spinal cord tissues are immediately dissected and placed on ice. Lumbar cords are then split and processed: homogenization and digestion for assessment of iron levels; RNA isolation for assessment of FTH1 gene expression; and fixation for immunohistochemical analysis. Blood is collected by cardiac puncture for iron and biomarker analyses. Skeletal muscle, spleen, heart and liver samples are also harvested at sacrifice for measurements of gene expression and immunohistochemical analyses. The spleen is split in half: the first half is flash frozen for immunohistochemical analyses, and the second half is processed to isolate macrophages using the EasySep Mouse F4/80 immunomagnetic positive selection kit (StemCell Technologies), from which RNA is isolated for gene expression analyses.

Iron measurement in the lumbar cords. At autopsy, the lumbar cords are collected, weighed, and digested in concentrated hydrochloric acid. Tissue iron concentrations (micrograms of iron per gram of tissue) are determined spectrophotometrically using a kit from Diagnostic Chemical Limited (Charlotteown, PE, Canada), with modifications for a microtiter plate assay.

Gene Expression. RNA isolation and gene expression analysis is performed as described in Example 8 above.

Immunohistochemical analysis. Organ samples are embedded in OCT compound and flash frozen. Frozen samples are sectioned on a cryostat prior and stained for H-Ferritin, and L-Ferritin. Sections are also stained with H&E for assessment of inflammation.

Statistical analysis. Data are presented as mean #SEM or SD. Differences between ID+6OHDA lesioned mice that are treated with vehicle, and ID+6OHDA lesioned mice that are treated with oligopeptides FT-006, FT-007, and FT-008, are analyzed using the original data or transformed data normalized to vehicle control (% of control). The outer fence is set at two standard deviations to analyze outliers per group. To correct for multiple testing, a one-way Bonferroni multiple-comparison test for a normal distribution and Kruskal-Wallis non-parametric test is applied for small number of animal samples. A p-value of <0.05 is considered significant.

Although the present disclosure has been described in some detail by way of illustration and example for purposes of clarity of understanding, it will be apparent to those skilled in the art that certain changes and modifications may be practiced in light of the above teaching. Therefore, the preceding examples should not be construed as limiting the scope of the present disclosure, which is delineated by the appended claims.

1. An isolated oligopeptide consisting of the amino acid sequence of Xm(R/D)EES(G/D)(E/K)Xn (Consensus No. 1), in which m and n are integers independently selected from the range of from 0-10, and each X, if present, is independently selected from any amino acid.

2. An isolated oligopeptide consisting of the amino acid sequence of XjREESDKPXk (SEQ ID NO:18), in which j is an integer selected from the range of from 0-10, k is an integer selected from the range of from 0-9, and each X, if present, is independently selected from any amino acid.

3. The isolated oligopeptide of claim 2, comprising the amino acid sequence of XgREESDKP (XhXi) (SEQ ID NO:19), in which Xg is proline or absent, XhXi is methionine and tyrosine or absent.

4. The isolated oligopeptide of claim 3, comprising the amino acid sequence of REESDKPMY (SEQ ID NO:6).

5. The isolated oligopeptide of claim 3, comprising the amino acid sequence of PREESDKP (SEQ ID NO:7).

6. The isolated oligopeptide of claim 1, comprising the amino acid sequence of REESGEL (SEQ ID NO:8).

7. The isolated oligopeptide of claim 1, comprising the amino acid sequence of REESDKPMY (SEQ ID NO:6), PREESDKP (SEQ ID NO:7), REESGEL (SEQ ID NO:8), REESGE (SEQ ID NO:1), REESGEP (SEQ ID NO:2), KEEDEESGE (SEQ ID NO:3), KPREESGE (SEQ ID NO:4), or LDEESGEP (SEQ ID NO:5).

8. The isolated oligopeptide of claim 7, wherein the oligopeptide is capable of increasing expression of ferritin heavy chain 1 (FTH1) mRNA by mammalian cells contacted with the oligopeptide.

9. A formulation comprising the isolated oligopeptide of claim 8, and at least one pharmaceutically acceptable excipient.

10. A formulation comprising the isolated oligopeptide of claim 8, and an oral delivery agent.

11. The formulation of claim 10, wherein the oral delivery agent comprises an absorption enhancer, a fatty acid, an enzyme inhibitor, polyethylene glycol, a mucoadhesive polymer, a cell penetrating peptide, or a combination thereof.

12. The formulation of claim 11, further comprising an enteric coating, liposomes, microspheres, and/or micro-/nano-particles.

13. An isolated nucleic acid encoding the oligopeptide of claim 7.

14. An expression vector comprising the nucleic acid of claim 13 in operable combination with a promoter.

15. A host cell comprising the isolated nucleic acid of claim 13 or the expression vector of claim 14.

16. A medicament comprising the formulation of claim 9.

17. A method for increasing expression of ferritin heavy chain 1 (FTH1) mRNA by mammalian cells, comprising contacting the mammalian cells with an effective amount of the formulation of claim 9 to increase FTH1 expression.

18. The method of claim 17, wherein the mammalian cells are intestinal epithelial cells, skeletal muscle cells, astrocytes, or macrophages.

19. The method of claim 17, wherein contacting is in vivo.

20. A method for increasing ferritin heavy chain 1 (FTH1) expression in a mammalian subject in need thereof, comprising administering to the subject an effective amount of the formulation of claim 9 to increase FTH1 expression.

21. A method for increasing serum ferritin concentration in a mammalian subject in need thereof, comprising administering to the subject an effective amount of the formulation of claim 9 to increase serum ferritin concentration.

22. A method for treating or preventing a disease or condition in a mammalian subject in need thereof, comprising administering to the subject an effective amount of the formulation of claim 9 to treat or prevent the disease or condition.

23. The method of claim 22, wherein the disease or condition is associated with an iron deficiency.

24. The method of claim 22, wherein the disease or condition is associated with anemia.

25. The method of claim 22, wherein the disease or condition is restless leg syndrome.

26. The method of claim 25, wherein the formulation is administered by mouth.

27. The method of claim 26, wherein the formulation is administered enterically.

28. The method of claim 26, wherein the formulation is administered by a buccal, a sublabial, or a sublingual route.

29. The method of claim 20, wherein the mammalian subject is a human subject.

30. The method of claim 29, wherein the human subject does not have cancer.


SEQUENCE LISTING (19 sequences)

SEQ ID NO: 1 - protein, 6 aa, synthetic construct
REESGE

SEQ ID NO: 2 - protein, 7 aa, synthetic construct
REESGEP

SEQ ID NO: 3 - protein, 9 aa, synthetic construct
KEEDEESGE

SEQ ID NO: 4 - protein, 8 aa, synthetic construct
KPREESGE

SEQ ID NO: 5 - protein, 8 aa, synthetic construct
LDEESGEP

SEQ ID NO: 6 - protein, 9 aa, synthetic construct
REESDKPMY

SEQ ID NO: 7 - protein, 8 aa, synthetic construct
PREESDKP

SEQ ID NO: 8 - protein, 7 aa, synthetic construct
REESGEL

SEQ ID NO: 9 - skipped

SEQ ID NO: 10 - DNA, 24 bp, synthetic construct
  source 1..24: mol_type = other DNA
taaagaactgggtgaccacgtgac

SEQ ID NO: 11 - DNA, 24 bp, synthetic construct
  source 1..24: mol_type = other DNA
aagtcagcttagctctcatcaccg

SEQ ID NO: 12 - DNA, 24 bp, synthetic construct
  source 1..24: mol_type = other DNA
tggccatggagaagaacctgaatc

SEQ ID NO: 13 - DNA, 23 bp, synthetic construct
  source 1..23: mol_type = other DNA
gctttccaggaagtcacagagat

SEQ ID NO: 14 - DNA, 23 bp, synthetic construct
  source 1..23: mol_type = other DNA
gtaatgatcagtcaacgggggac

SEQ ID NO: 15 - DNA, 24 bp, synthetic construct
  source 1..24: mol_type = other DNA
ccagcaagcttgcaaccttaacca

SEQ ID NO: 16 - DNA, 19 bp, synthetic construct
  source 1..19: mol_type = other DNA
ggagttgtatgcctcctac

SEQ ID NO: 17 - DNA, 18 bp, synthetic construct
  source 1..18: mol_type = other DNA
gagatattctgccatgcc

SEQ ID NO: 18 - protein, 9 aa, synthetic construct
  VARIANT 1: note = Absent or present in repeats of up to 10; each X, if present, is independently selected from any amino acid
  VARIANT 9: note = Absent or present in repeats of up to 9; each X, if present, is independently selected from any amino acid
XREESDKPX

SEQ ID NO: 19 - protein, 10 aa, synthetic construct
  VARIANT 1: note = Proline (P) or absent
  VARIANT 9..10: note = Methionine and tyrosine (MY) or absent
XREESDKPXX